(19) United States
(12) Patent Application Publication
(10) Pub. No.: US 2010/0179103 A1
(43) Pub. Date: Jul. 15, 2010

(54) CURCUMIN CYCLODEXTRIN COMBINATION FOR PREVENTING OR TREATING VARIOUS DISEASES

(76) Inventor: Ketan Desai, Easton, PA (US)

Correspondence Address: Ketan Desai, 1175 Spring Road, Easton, PA, 18040, US

(21) Appl. No.: 12/488,781

(22) Filed: Jun. 22, 2009

Related U.S. Application Data
(60) Provisional application No. 61/135,443, filed on Jul. 21, 2008.

(51) Int. Cl. A61K 47/48; C08B 37/16; A61P 25/00; A61P 25/28; A61P 29/00; A61P 37/08; A61P 11/06; A61P 35/00; A61P 11/00; A61P 17/00; A61P 9/00; A61P 3/06; A61P 1/00

(52) U.S. Cl. 514/58; 536/103

(57) ABSTRACT

Curcumin has shown anti-inflammatory and anti-angiogenic properties that could be useful in treating various diseases such as those of rheumatology and oncology. However, curcumin is very poorly absorbed and has a very low bioavailability. This patent describes a method of increasing the delivery of curcumin by complexing it with cyclodextrins. Cyclodextrins are well known in the food industry and have been used to carry other drugs to increase bioavailability. The new combination of cyclodextrins and curcumin has been tested in pre-clinical inflammation models where it has demonstrated efficacy superior to both the positive control and curcumin.

This application is based on U.S. Provisional Patent No. 61/135,443 Application filed Jun. 22, 2009, the entire contents of which are incorporated herein.

### FIELD OF THE INVENTION

The present invention relates to method of use patent for the complex of curcumin and cyclodextrin to treat diseases affecting various systems, in humans and animals. The diseases include neurological diseases such as Alzheimers, autoimmune or inflammatory or allergic diseases such as asthma and rheumatoid arthritis, oncologic diseases, respiratory system diseases such as chronic obstructive lung disease, dermatologic diseases, cardiovascular diseases such as hyperlipidemia and coronary artery disease, gastrointestinal, hepatic and pancreatic diseases such as inflammatory bowel disease, metabolic diseases such as diabetes, urologic, and infectious diseases, and for wound healing. Administration can be oral, topical, parenteral, nasal, inhalational, or suppository.

### BACKGROUND OF THE INVENTION

Turmeric and turmeric extracts have long been active ingredients in compositions for oral and topical application to treat diseases and conditions.

Curcuma longa (Fam. Zingiberaccae), or turmeric, is a spicy plant that is a common ingredient in curry powder. Turmeric has a strong yellow color that is useful in providing a pleasing color to foods such as prepared mustard. Turmeric has antimicrobial properties that can help preserve food and prevent spoilage.

Turmeric is one of the oldest herbs in Ayurveda materia medica, and has been used in Ayurveda medicine internally as a tonic, to settle an upset stomach, and as a blood purifier. Turmeric has been applied topically to wounds to stop bleeding, speed healing and reduce scaring. Ground turmeric has been used as a topical salve, particularly in India, to prevent and treat a variety of skin diseases and conditions.

The significance of turmeric in medicine has changed in modern times with the scientific observation of turmeric's therapeutic properties. The same ground dried rhizome of Curcuma longa, which has been used for centuries as a spice, food preservative and coloring agent, has been found to be a rich source of beneficial phenolic compounds, turmerin peptide, and curcuminoids. Curcuminoids have scientifically documented anti-oxidant, anti-inflammatory, anti-bacterial, anti-fungal, antiparasitic, anti-mutagen, anti-cancer and detoxification properties. Curcuminoids are recognized for their broad biological activity and safety of use. Significant data are available on the safety, toxicity, dose range, pharmacokinetics, and other biological properties of turmeric and its components, including curcuminoids and turmerin peptide. Turmeric components are well tolerated while providing anti-oxidant benefits, inhibit microbial growth, inhibit several enzymes, and inhibit abnormal cell growth in studies of cells, animals, and humans.

Studies have shown that turmeric components can inhibit enzymes associated with disease states. Extensive in vitro and in vivo testing has shown that turmeric inhibits chemically-induced epidermal ornithine decarboxylase activity, epidermal DNA synthesis, and the promotion of skin tumors in mice (Conney, A. H. et al., Adv. Enzyme Regul., 31:385-396, 1991; Huang, M. T. et al., Cancer Res., 48:5941-5945, 1988; Lu, Y. P. et al., Carcinogenesis, 14:293-297, 1993; Azuine, M. A., Bhide, S. V., Nutr Cancer, 17:77-83, 1992). Further studies suggest that turmeric also reduces arachidonic acid-induced rat paw and mouse skin edema and markedly inhibits epidermal lipoxygenase and cyclooxygenase activity in vitro (Rao, T. S. et al., Indian J. Med. Res., 75:574-578, 1982; Conney, A. H. et al., Adv. Enzyme Regul., 31:385-396, 1991; Huang, M. T. et al., Cancer Res., 48:5941-5945, 1988). Phosphorylation events can also be influenced by curcumin, as it has been reported that curcuminoids inhibit protein kinase C activity induced by 12-O-tetradecanoyl-phorbol-13-acetate in NIH 3T3 cells (Liu, J. Y., Lin, S. J., and Lin, J. K., Carcinogenesis, 14:857-861).

Turmeric can fight against microbial infections and parasitic infestations. In humans, ingestion of turmeric has been used to treat biliary infections where it demonstrates bacteriostatic or bacteriocidal effects against organisms involved in cholecystitis (Ramprasad, C. et al., Ind. J. Phys. and Pharm., 1:136-143, 1957; Lutumski, J. et al., Planta Med., 26:9-19, 1974). Topical application of a turmeric paste for the treatment of scabies has also shown good results (Charles, V., Charles, S. X., Trop. Geogr. Med., 44:178-181, 1992).

Their potential use of turmeric and turmeric extracts in the prevention of cancer and in the treatment of infection with human immunodeficiency virus (HIV) are the subject of intensive laboratory and clinical research. It has been shown that curcuminoids decreased p24 antigen production in acutely or chronically infected cells with HIV-1, a paradigm of anti-viral activity (Li, C. J. et al., Proc. Natl. Acad. Sci. USA, 90:1839-1842, 1993). The addition of turmeric to the diet has been shown to inhibit azoxymethanol-induced colonic epithelial cell proliferation and focal areas of dysplasia (Huang, M. T. et al., Cancer Letters, 64:117-121, 1992). It has also been shown to interfere with the formation of covalent carcinogen-DNA adducts (Mukudan, M. A. et al., Cardnogenesis, 14:493-496, 1993).

Fat metabolism is likewise influenced by curcumin. It can render bile non-lithogenic in mice (Hussain, M. S. et al., Indian J. Med. Rcs., 96:288-291, 1992). Curcuminoids can reduce the production of PMA-induced lipid peroxidation and 8-OH-deoxyguanosine formation in mouse fibroblast cells (Shih, C. A., and Lin, J. K., Carcinogenesis, 14:709-712, 1993). Oral administration of curcuminoids in human volunteers has been shown to significantly decrease the level of serum lipid peroxides (33%), increase HDL cholesterol (29%), and decrease total serum cholesterol (11.63%) (Soni, K. B, Kuttan R., Indian J. Phys. Pharmacol., 36:273-275, 1992).

Curcumin can moderate the immune system as well as smooth muscle cell proliferation. Activation responses to phytohemagglutinin and mixed lymphocyte reaction were reduced in human peripheral blood mononuclear cells in the presence of curcuminoids. Furthermore, curcuminoids inhibited the proliferation of rabbit vascular smooth muscle cells stimulated by fetal calf serum. Curcuminoids had a greater inhibitory effect on platelet derived growth factor-stimulated proliferation than on serum-stimulated proliferation (Huang, H. C. et al., Eur. J. Pharmacol., 221:381-384, 1992).

The anti-inflammatory properties of curcuminoids were shown to inhibit the 5-lipoxygenase activity in rat peritoneal neutrophils as well as the 12-lipoxygenase and the cyclooxygenase activities in human platelets (Ammon, H. P. T. et al, J. Ethopharmacol., 38:113-119, 1993). Curcuminoids had no significant effect on quercetin-induced nuclear DNA damage, lipid peroxidation and protein degradation, and thus has the unique potential of acting as both pro- and antioxidants, depending on the redox state of their biological environment (Saura, C. et al., Cancer Letters, 63:237-241, 1992). Administration of curcuminoids in mice exhibited antioxidant properties by significantly reducing the scavenging of peroxides and other activated oxygen species, (Soudamini, K. K. et al., Indian J. Phys. Pharmacol. 36:239-243, 1992).

Pathogenesis of diseases like Alzheimer's show mounting evidence of oxidative damage and inflammatory factors. Unfortunately, despite strong epidemiology and rationale, antioxidant and NSAED approaches to these age-related diseases have generally not been successful in the clinic. For example, vitamin E has failed in trials for Alzheimer's and heart disease prevention, while COX inhibitors have failed for Alzheimer treatment and been dropped for prevention efforts with traditional antioxidants (selenium, vitamin E, β carotene), estrogens, and COX-2 inhibitors. The demographics of aging population drive an urgent need for suitable alternatives for prevention and possible treatment of one or more of the chronic diseases of aging.

As a tumeric extract, curcumin is the yellow in yellow curries and is used as a food additive, for example, in yellow mustard. Like the “wonder drug” aspirin, which remains one of our few successful preventive agents, the long-term health potential of curcumin has a substantial history and a relatively well-established scientific basis. It has been identified as a major bioactive agent in an empirically developed system of traditional Indian medicine.

Curcumin (diferulomethane) is not only a potent natural antioxidant and anti-inflammatory agent, acting on NFkB and AP-I regulated pro-inflammatory mediators including COX-2, iNOS, il-1 and TNFα, but has multiple useful activities and has shown therapeutic potential in many pre-clinical in-vitro and animal models for diseases, often related to aging. These include cancers (colon, prostate, breast, skin, leukemia, etc.) (Agarwal et al., 2003), prion disease (Caughey et al., 2003), atherosclerosis (Miguel et al., 2002; Ramaswami et al., 2004), stroke (G. Sun, personal comm.), CNS alcohol toxicity (Rajakrishnan et al., 1999), traumatic brain injury (F. Gomez-Pinilla, UCLA personal comm.), Huntington's disease (MF Chesselet, personal comm.), Marie-Charcot Tooth (J. Lupski, personal comm.), multiple sclerosis (EAE), and Alzheimer's disease.

Curcumin can block aggregation of Aβ and other amyloid-forming peptides to toxic fibrils and oligomers; chelate metals that cause lipid and protein and DNA oxidative damage to the brain; inhibit aberrant inflammation through AP-I and NFkB transcription; stimulate beneficial microglial phagocytosis like the amylid vaccine to clear amyloid out of brain; and inhibit production of BACE under conditions of oxidative damage and inflammation, thus limiting Aβ production. Broad Spectrum efficacy for age-related disease is accentuated by positive life extension data (Kitani et al., 2004). Based on its outstanding safety profile and efficacy in multiple disease models with oxidative damage and inflammatory factors, curcumin has shown excellent potential for disease pathogenesis in Alzheimer models. Curcumin is on the short list of useful agents for cancer chemoprevention under development by the National Cancer Institute (NCI), which put curcumin through the National Toxicology Program and pre-clinical safety and efficacy trials (KelJoff et al., 1996; Chainani-Wu, 2003). Curcumin has passed several phase II trials for cancer and is currently in further clinical trials for cancers at multiple sites in the US and abroad (MD Anderson web site).

Curcumin's structure resembles that of amyloid binding compounds. Amyloid dyes like Congo Red (CR) are known to bind via planar hydrophobic groups with appropriately spaced charge, and to suppress β-amyloid and other β-sheet-dependent peptide aggregation and toxicity. The Congo Red analogue, Chrysamine G, is more brain permeant and retains CR's amyloid binding properties. Curcumin shares the 19 angstrom CR spacing between its polar phenol groups; is readily brain permeant; and binds amyloid peptides, inhibiting their aggregation and toxicity in vitro. Curcumin effectively reduces amyloid accumulation in vivo in APP Tg mice. Because CR's anti-amyloid binding is generic and potentially relevant to other β-sheet intraneuronal aggregates including Huntington, a-synuclein, prions and tau, curcumin's anti-amyloid activity may be relevant beyond extracellular amyloid to intraneuronal aggregates. In fact, curcumin is one of the most effective anti-prion compounds ever tested in vitro, although it did not work in vivo with oral dosing of unstated formulation (Caughey et al., 2003). This raises the limitations of curcumin oral bioavailability, the subject of the present invention. The benefits of curcumin as a treatment for multiple diseases with aggregating amyloid proteins and other CAG repeat disorders are being established, and its efficacy in treating stroke, head trauma, metabolic syndrome, and many other conditions, including some forms of cancer and arthritis, as well as in promoting wound healing, is also beginning to be understood. All of these therapeutic applications are limited, however, because of poor intestinal absorption.

Nuclear factor-.kappa B (NFkB) is very important to cell function and is critical to the immune system and cancer biology. Agents that can suppress the activation of nuclear factor kappa B (NFkB) and activator protein-1 (AP-1), may be able to block tumorigenesis and inflammation. Curcumin blocks tumor necrosis factor (TNF) induced activation of NFkB in a concentration and time dependent manner. The effects are not cell type specific because it blocked TNF induced NFkB activation in a variety of cells. The NFkB-dependent reporter gene transcription activated by TNF was also suppressed.

Rel/NFkB transcription factors are a family of structurally related eukaryotic transcription factors that are involved in the control of a large number of normal cellular processes, such as immune and inflammatory responses, developmental processes, cellular growth, and apoptosis. In addition, these factors are active in a number of disease states, including cancer, arthritis, chronic inflammation, asthma, neurodegenerative, and heart disease. Rel/NFkB transcription factors include a collection of proteins conserved from the fruit fly Drosophila melanogaster to humans. Among the commonly used model organisms, these transcription factors are notably absent in yeast and the nematode Caenorhabditis elegans. In part this may be because one of the primary roles of these factors is to control a variety of physiological aspects of immune and inflammatory responses. A pathway similar to the Rel/NFkB signaling pathway may also control certain defense responses in plants.

In most cells NFkB is present as a latent, inactive, I kappa B bound complex in the cytoplasm. When a cell receives any of a multitude of extracellular signals, NFkB rapidly dissociates from 1 kappa B, enters the nucleus and activates gene expression. Therefore a key step for controlling NFkB activity is the regulation of the interaction of I kappa B and NFkB. Many of the molecular details of this control mechanism are now understood. Almost all of the signals that lead to the activation of NFkB converge on a high molecular weight complex that contains a serine specific I kappa B kinase (IKK). IKK is an unusual kinase in that in most cells IKK contains at least three distinct subunits: IKK alpha, IKK beta and IKK gamma. IKK alpha and IKK beta are related catalytic kinase subunits, and IKK gamma is a regulatory subunit that serves as a control mechanism for the catalytic subunits. In the classical or canonical pathway, activation of IKK complex leads to the phosphorylation by IKK beta. of two specific serines near the N terminus of IkB, which targets IkB for ubiquitination and degradation by the proteasome. In the non-canonical pathway, the p100-RelB complex is activated by IKK alpha mediated phosphorylation of the C-terminal region of p100, which leads to degradation of the p100 IkB-like C-terminal sequences to generate p52-RelB. In either pathway, the unmasked Rel/NFkB complex can then enter the nucleus to activate target gene expression. In the canonical pathway, one of the target genes activated by NFkB is that which encodes I kappa B. Newly synthesized I kappa B can enter the nucleus, remove NFkB from DNA, and export the complex back to the cytoplasm to restore the original latent state. Thus, the activation of the NFkB pathway is generally a transient process, lasting from 30 to 60 minutes in most cells.

In some normal cells, such as B cells, some T cells, Sertoli cells and some neurons, NFkB is constitutively located in the nucleus. In addition, in many cancer cells, including breast cancer, colon cancer, prostate cancer, lymphoid cancers, and probably many others, NFkB is constitutively active and located in the nucleus. In some cancers, this is due to chronic stimulation of the IKK pathway, while in other cells, such as some Hodgkin's and diffuse large B-cell lymphoma cells, the gene encoding IkB is sometimes mutated and defective. Moreover, several human lymphoid cancer cells have mutations or amplifications of genes encoding Rel/NFkB transcription factors, which may enable them to accumulate in or rapidly and repeatedly cycle through the nucleus. It is thought that continuous nuclear Rel/NFkB activity protects cancer cells from apoptosis and in some cases stimulates their growth. Therefore, many current anti-tumor therapies seek to block NFkB activity as a means for inhibiting tumor growth or sensitizing the tumor cells to more conventional therapies, such as chemotherapy.

Diseases Associated with NFkB:

| Disease | | Reference |
|---|---|---|
| 1 | Aging | Reuter et al., 2003 |
| 3 | Cardiac Hypertrophy | Purcell & Molkentin, 2003 |
| 4 | Muscular Dystrophy (type 2A) | Baghdiguian et al., 1999 |
| 5 | Catabolic disorder | Holmes-McNary, 2002 |
| 6 | Diabetes, Type 1 | Ho & Bray, 1999 |
| 7 | Diabetes, Type 2 | Yuan et al., 2001 |
| 8 | Hypercholesterolemia | Wilson et al., 2000 |
| 9 | Atherosclerosis | Ross et al., 2001 |
| 10 | Heart Disease | Valen et al., 2001 |
| 11 | Ischemia/reperfusion | Toledo-Pereyra & Lopez- |
| | | Neblina, 2002 |
| 12 | Angina Pectoris | Ritchie, 1998 |
| 13 | Pulmonary Disease | Christman et al., 2000 |
| 14 | Acid-induced Lung Inury | Madjdpour et al., 2003 |
| 15 | Renal Disease | Guijarro & Egido, 2001 |
| 16 | Leptospiriosis renal disease | Yang et al., 2001 |
| 17 | Gut Diseases | Neurath et al., 1998 |
| 18 | Skin Diseases | Bell et al., 2003 |
| 19 | Incontinentia pigmenti | Courtois & Israel A, 2000 |
| 20 | Asthma | Pahl & Szelenyi, 2002 |
| 21 | Arthritis | Roshak et al., 2002 |
| 22 | Crohns Disease | Pena & Penate, 2002 |
| 23 | Ocular Allergy | Bielory et al., 2003 |
| 24 | Appendicitis | Pennington et al., 2000 |
| 25 | Pancreatitis | Weber & Adler, 2001 |
| 26 | Periodonitis | Nichols et al., 2001 |
| 27 | Inflammatory Bowel Disease | Dijkstra et al., 2002 |
| 28 | Sepsis | Wratten et al., 2001, |
| | | Abraham, 2003 |
| 29 | Silica-induced | Chen & Shi, 2002 |
| 30 | AIDS (HIV-1) | Hiscott et al., 2001 |
| 31 | Autoimmunity | Hayashi & Faustman, 2000 |
| 32 | Lupus | Kammer & Tsokos, 2002 |
| 33 | Neuropathological Diseases | Cechetto, 2001, Mattson & |
| | | Camandola, 2001 |
| 34 | Sleep apnoea | Lavie, 2003 |
| 35 | Alzheimers Disease | Mattson & Camandola, 2001 |

Constitutive Activation of NFkB in Human Cancer Cells

| Cancer Type | | Reference |
|---|---|---|
| 1 | Breast | Nakshatri et al., 1997; Sovak et al., |
| | | 1997 |
| 2 | Cervix | Nair et al., 2003 |
| 3 | Ovary | Dejardin et al., 1999; Huang et al., 2000 |
| 4 | Vulva | Seppanen & Vihko, 2000 |
| 5 | Prostate | Huang et al., 2001b; Palayoor et al., |
| | | 1999 |
| 6 | Kidney | Oya et al., 2001 |
| 7 | Liver | Tai et al., 2000 |
| 8 | Pancreas | Wang et al., 1999; Sclabas et al., 2003 |
| 9 | Esophygeal/gastric | Sutter et al., 2004 |
| 10 | Stomach | Sasaki et al., 2001 |
| 11 | Colon | Lind et al., 2001 |
| 12 | Thyroid | Visconti et al., 1997 |
| 13 | Melanoma | Yang & Richmond, 2001 |
| 14 | Head and neck | Ondrey et al., 1999 |
| 15 | Cylindromatosis | Kovalenko et al., 2003; Brummelkamp |
| | | et al., 2003; Tropmpouki et al., 2003 |
| 16 | Oral carcinoma | Nakayama et al., 2001 |
| 17 | Astrocytoma/glioblastoma | Hayahsi et al., 2001 |
| 18 | Neuroblastoma | Bian et al., 2002 |
| 19 | Acute lymphoblastic leukemia | Kordes et al., 2000 |
| 20 | Acute myelogenous leukemia | Guzman et al., 2001 |
| 21 | Acute T-cell leukemia | Arima & Tei, 2001 (HTLV-1) |
| 22 | Chronic lymphocytic leukemia | Furman et al., 2000 |
| 23 | Burkitts Lymphoma (EBV) | Knecht et al., 2001 |
| 24 | Mantle cell lymphoma | Martinez et al., 2003 |
| 25 | Multiple myeloma | Berenson et al., 2001 |
| 26 | Diffuse large B-cell lymphoma | Davis et al., 2001; Shaffer et al., 2002 |
| 27 | Hodgkin's lymphoma | Bargou et al., 1996, 1997; Staudt, 2001 |

Due to its action on NFkB and its anti-oxidative properties, in addition to its many other effects, curcumin has been proposed to treat various diseases mentioned in [0016] and [0017]. Although many groups have come up with theoretical ideas for improving absorption of curcumin, most have involved entirely in vitro studies, probably because of the difficulty in measuring curcumin and its metabolites in tissue.

Although curcumin is an effective medication in multiple animal models for human diseases when given with food at high doses (typically 2,000-5,000 ppm in diet in cancer trials), the current dogma is that it is so poorly bioavailable that it cannot be used for treatment outside the colon in humans. Curcumin is very hydrophobic and typically is not dissolved when delivered as a powder extract in common nutraceuticals. Most curcumin activities require 100-2,000 nanomolar (0.1-2 micromolar) levels in vitro, but current supplements result in negligible, low nanomolar blood levels (see Sharma et al., 2004). R. Sharma's group at Leicester has tried repeatedly and been unable to achieve significant blood levels beyond the low nanomolar range (Garcea G., Jones J D, Singh R., Dennison A R, Farmer P B, Sharma R A, Steward W P, Gescher A J, Berry D P Detection of curcumin and its metabolites in hepatic tissue and portal blood of patients following oral administration. Br J. Cancer. 2004 Mar. 8; 90 (5): 1011-5. PMBD: 14997198.) They and others conclude that delivery of effective concentrations of oral curcumin to systemic tissues (outside the GI tract) is “probably not feasible.” Most of the literature supports this view, leading the NCI to focus on colon cancer. In addition, curcumin has very poor bioavailability, with Tₘₐₓ of only 3-5 minutes

Curcumin has been extensively studied in various diseases ranging from phase Ito phase IIb. In Phase-I clinical studies, oral administration of curcumin is well tolerated at pharmacological concentration. However, as mentioned above, its bioavailability is very poor. Cheng et al conducted a phase I study in patients with Bowens Disease and found that doses up to 8000 mg/day for 3 months were well tolerated (Cheng, A L, et al Anticancer Res 2001. 21, 2895-2900). Serum concentrations peaked at 1-2 hours and then gradually declined. Maximum serum concentrations ranged from 0.5+/−0.11 mM at 4000 mg/day to 1.77+/−1.87 mM at 8000 mg/day. No difference was observed with repeated dosing (on day 30). Many other phase I and phase II studies have come to similar conclusions—that while there is a lot of scientific merit to the use of curcumin, its poor bioavailability limits its use (reviewed by Hatcher H et al, 2008).

Three factors limit curcumin absorption and need to be addressed: 1) rapid glucuronidation/sulfation of curcumin's phenolic hydroxyl groups and high “first pass” clearance; 2) curcumin is unstable in aqueous solution at pH 7 and above; and 3) curcumin is very hydrophobic and typically is not water soluble at acidic pH and when delivered as a dry powder in existing supplements. (Most of the curcumin is never absorbed and simply passes through the GI tract and is excreted.)

Solubilization is critical to prevent this, but curcumin requires pH 8.5 to dissolve completely. For this reason, cancer patients take huge doses, typically up to 8 gms a day. Diarrhea is a common side-effect. Garcea, G. et al. (2004) report that with patients taking 3.6 gms of curcumin a day (as a standard powder extract capsule supplied by Sabinsa Corporation), blood and liver levels achieved are negligible. They conclude that “the results suggest that doses of curcumin required to furnish hepatic levels sufficient to exert pharmacological activity are probably not feasible in humans.”

Curcumin is not soluble at acidic pH and breaks down when solubilized and diluted into water at neutral or alkaline pH (e.g., in the GI tract, after the small intestine), due to keto-enol transformations in the β-diketonebridge. In addition, curcumin is susceptible to rapid sulfation/glucuronidation. The major U.S. supplier, Sabinsa, has tried to make a more bioavailable form by adding Bioperine (pipeline) to inhibit glucuronidation. Such an approach is flawed, however, because most glucuronidation takes place in the upper GI tract, where the pH is acidic, and curcumin is not completely dissolved until pH 8.5 and higher. Even worse, inhibiting glucuronidation can cause serious health risks. Glucuronidation is protective against many toxins and involved in the metabolism of commonly used drugs. Most elderly patients are on multiple drugs, at levels likely to be unsafely altered by inhibition of glucuronidation.

Cyclodextrins are cyclic oligomers of glucose; these compounds form inclusion complexes with any drug whose molecule can fit into the lipophile-seeking cavities of the cyclodextrin molecule. They make an excellent vehicle for carrying other molecules, especially insoluble compounds such as curcumin.

Cyclodextrin can be a-, β-, or gamma-cyclodextrin. Some of the cycldextrins are hydroxypropyl, hydroxyethyl, glucosyl, maltosyl and maltotriosyl derivatives of (3-cyclodextrin, carboxyamidomethyl-β-cyclodextrin, carboxymethyl-p-cyclodextrin, hydroxypropyl-β-cyclodextrin and diethylamino-β-cyclodextrin. In the composition according to this invention hydroxy-p-cyclodextrin is preferred but others can be used. The substituted-cyclodextrins may also be suitable, including hydroxypropyl, hydroxyethyl, glucosyl, maltosyl and maltotriosyl derivatives of y-cyclodextrin.

Alpha-cyclodextrin contains six glucopyranose units; beta-cyclodextrin contains seven glucopyranose units, and gamma cyclodextrin contains eight glucopyranose units. The molecules are believed to form a truncated cone having a core opening of 4.7-5. 3 A, 6.0-6. 5 A and 7.5-8. 3 A in alpha, beta, or gamma cyclodextrin respectively. The composition according to this invention may comprise a mixture of two or more of the alpha, beta, or gamma cyclodextrins. Usually, however the composition according to this invention will comprise only one of the cyclodextrins.

There are several patents that indicate that complexes of cyclodextrin with drug substances improve the side effect profile of the drug substance. Szejtli et al., U.S. Pat. No. 4,228,160, disclosed that the frequency and severity of gastric and duodenal erosion and ulceration in rats caused by indomethecin is improved in an oral formulation of a complex of beta-cyclodextrin and indomethacin in a 2:1 ratio, but is not improved and in fact worsens in the same oral formulation of a complex of beta-cyclodextrin and indomethacin in a 1:1 ratio.

Shimazu et al., U.S. Pat. No. 4,352,793, discloses that a formulation wherein bencyclane fumarate, an anti-convulsive compound with beta.-cyclodextrin or gamma.-cyclodextrin yields a complex in which the bencyclane fumarate is an inclusion compound. These complexes, when formulated as a liquid suitable for oral administration were claimed to be less irritating in an isotonic buffered pH 7 solution when administered as drops to the eyes of rabbits, as compared to bencyclane fumarate drops at the same drug concentration in an inactive vehicle. Shimazu et al., also disclosed that similar complexes dissolved in rabbit blood in-vitro yielded reduced hemolysis as compared to equal concentrations of bencyclane fumarate alone mixed with rabbit blood.

Masuda et al., U.S. Pat. No. 4,478,811, disclose ophthalmic formulations of beta or gamma-cyclodextrin complexes of the nonsteroidal anti-inflammatory compound fluorobiphenylacetic acid that are less irritating and painful than the same formulations of fluorobiphenyl acetic acid alone. Uekama et al., U.S. Pat. No. 4,565,807, disclose complexes of alpha, beta, and gamma cyclodextrin, piprofen and a pharmaceutically acceptable base. Piprofen is an analgesic and anti-inflammatory compound, which is bitter and can cause irritation to the gastrointestinal tract. The complexes disclosed in the patent have improved less bitter taste and are less irritating to the gastrointestinal tract than is the uncomplexed compound piprofen. No preparations suitable for intravenous injection were disclosed.

Lipari, U.S. Pat. No. 4,383,992, disclose topical and ophthalmic solutions comprising a number of different steroid-related compounds including corticosteroids, androgens, anabolic steroids, estrogens, and progestagens complexed with beta cyclodextrin. None of the cyclodextrin compounds disclosed by Lipari are substituted or amorphous cyclodextrins. In addition, none or the steroid related compounds disclosed by Lipari are 5 beta steroids.

Pitha et al., U.S. Pat. No. 4,596,795 disclose complexes containing amorphous hydroxypropyl-beta cyclodextrin and sex hormones, particularly testosterone, progesterone and estradiol as lyophilized powders. These tableted complexes are disclosed as appropriate for administration sublingually or buccally with absorption occurring across the corresponding mucosal membrane. None is administered in solution parenterally. In addition none of the steroid related compounds disclosed by Pitha are 5 beta steroids.

Pitha et al., U.S. Pat. No. 4,727,064, disclose complexes containing water soluble amorphous substituted cyclodextrin mixtures and drugs with substantially low water solubility which may be lyophilized and the lyophilized powder formed into dosage forms suitable for absorption trans-mucosally across the oral, buccal or rectal mucosa. The solutions of amorphous, water soluble cyclodextrin alone and not in a complex with a drug substance are disclosed as nonirritating topically, and having low toxicity, both systemic and local, when applied parenterally. These solutions of substituted cyclodextrin alone were tested and shown to be non-lethal when substantial amounts of the cyclodextrin solution were administered intra-peritoneally in mice. A number of categories of drugs are disclosed in Pitha for complex with cyclodextrin derivatives and include inter alia vitamins, salts of retinoic acid, spironolactone, antiviral agents, diuretics, anticoagulants, anticonvulsant and anti-inflammatory agents. Significantly, Pitha, while disclosing that aqueous solutions of 50% cyclodextrin may be used for the purpose of determining solubility of drugs in such solutions does not indicate that such solutions are suitable for intravenous administration. No attempt is made to qualify the solution as to its pyrogenicity. The claimed compositions of matter in the reference contain only cyclodextrin and drug. Liquid or semi-liquid compositions of matter, which are claimed in the reference, appear to be made of cyclodextrins with higher degrees of substitution with hydroxypropyl groups. These cyclodextrins are themselves semi-solid or liquids according to the reference. Thus no aqueous formulations of water, cyclodextrin and drug are disclosed or claimed as suitable for parenteral administration in the reference.

Bekers et al. (1989) describe the investigation of stabilization of mitomycin-C and several related mitomycins by formation of a complex with cyclodextrin. The authors indicate that at the pH ranges studied alpha and beta cyclodextrin as well as heptakis-(2,6,-di-O-methyl)-beta cyclodextrin and dimethyl beta cyclodextrin, have no influence on stabilization of mitomycin-C pH dependant degradation. Gamma Cyclodextrin is reported as having measurable stabilizing effect on mitomycin in acidic media at pH's above 1.

Bodor, U.S. Pat. No. 5,024,998, and Bodor, U.S. Pat. No. 4,983,586, disclose a series of compositions comprising complexes of hydroxypropyl-beta cyclodextrin (HPCD) complexed to a difficult to solubilize drug, and HPCD complexed to a drug which has first been complexed to a specific class of drug carriers characterized as redox drug carriers. The complex of drug and redox carrier is itself difficult to solubilize and is highly lipophilic due to the presence of pyridine derivatives as part of the redox carrier complex. Bodor U.S. Pat. No. 5,024,998 and U.S. Pat. No. 4,983,586 further claim that a solution of 20 to 50% hydroxypropyl beta cyclodextrin and lipophilic drug-redox carrier complex, or 20 to 50% hydroxypropyl beta cyclodextrin and lipophilic and/or water labile drug is useful in a method of decreasing the incidence of precipitation of a lipophilic and/or water labile drug occurring at or near the injection site and/or in the lungs or other organs following parenteral administration. Significantly the Bodor references attribute the precipitation and organ deposition problems associated with parenteral administration of lipophilic drugs to the effects of organic solvents used to solubilize the drug in the parenteral vehicle. The Bodor references additionally state that drugs which are particularly useful in the parenteral composition and methods disclosed therein are those which are relatively insoluble in water but whose water solubility can be substantially improved by formulation with 20 to 50% of the selected cyclodextrin, e.g. HPCD, in water. Thus it is quite clear that the Bodor references are directed to prevention of the phenomenon of precipitation of insoluble drugs and insoluble drug-carrier complexes.

U.S. Pat. No. 5,824,668 discloses the composition of 5 beta steroid with cyclodextrin suitable for parenteral administration for treating various diseases.

Muller et al. (1992) described the complex formation of digitoxin with beta and gamma cyclodextrins. Uekama et al. (1983) described the inclusion complexes of the digitalis glycosides digitoxin, digoxin, and methyl digoxin with three cyclodextrins, the alpha, beta, and gamma homologues in water and in the solid state. Solid complexes in a molar ratio of 1:4 of the digitalis glycosides with gamma cyclodextrin were prepared and their in-vivo absorption examined. The rapidly dissolving form of the gamma cyclodextrin complex significantly increased plasma levels of digoxin (approximately 5.4-fold) after oral administration to dogs. Ueda et al. (1999) examined the complex formation of digitoxin with delta cyclodextrin and observed enhanced solubility. Okada and Koizumi (1998) studied the complex formation of digitoxin and digoxin with modified beta cyclodextrins.

U.S. Pat. No. 6,407,079 discloses the pharmaceutical compositions comprising inclusion compounds of sparingly water-soluble or water labile drugs with beta cyclodextrin ethers or beta cyclodextrin esters and the process for the preparation of such compositions. The patent claims cardiac glycosides as one of the types drugs for the treatment of cardiac disorders. The patent further states that molar ratio of the drug to the cyclodextrin derivative is from about 1:6 to 4:1. The patent claims injectable formulations with 0.45 micron filtering and sterilization. According to the patent document, the patent was filed in 1988 and was awarded in 2002. However, the complexation of digitoxin and digoxin with beta and gamma cyclodextrins was disclosed to the public by the inventors in 1992 (Muller et al. 1992).

The following is a list of approved drugs using cyclodextrins in combination:

| Cyclodextrin/Drug | | | |
|---|---|---|---|
| Region | Administration | Trade name | |
| Alprostadil (PGE₁) | Intravenous | Prostavastin, | Europe, |
| | | Caverject, Edex | Japan, |
| | | | United |
| | | | States |
| Cefotiam hexetil HCl | Oral | Pansporin T | Japan |
| Limaprost | Oral | Opalmon, Prorenal | |

-Cyclodextrin

| | | | |
|---|---|---|---|
| Benexate | Oral | Ulgut, Lonmiel | Japan |
| Dexamethasone | Dermal | Glymesason | Japan |
| Iodine | Topical | Mena-Gargle | Japan |
| Nicotine | Sublingual | Nicorette | Europe |
| Nimesulide | Oral | Nimedex, Mesulid | Europe |
| Nitroglycerin | Sublingual | Nitropen | Japan |
| Omeprazole | Oral | Omebeta | Europe |
| Dinoprostone (PGE₂) | Sublingual | Prostarmon E | Japan |
| Piroxicam | Oral | Brexin | Europe |
| Tiaprofenic acid | Oral | Surgamyl | Europe |

2-Hydroxypropyl--cyclodextrin

| | | | |
|---|---|---|---|
| Cisapride | Rectal | Propulsid | Europe |
| Hydrocortisone | Buccal | Dexocort | Europe |
| Indomethacin | Eye drops | Indocid | Europe |
| Itraconazole | Oral, I.V | Sporanox | Europe, |
| | | | United |
| | | | States |
| Mitomycin | Intravenous | Mitozytrex | United |
| | | | States |

Randomly methylated -cyclodextrin

| | | | |
|---|---|---|---|
| 17-Oestradiol | Nasal spray | Aerodiol | Europe |
| Chloramphenicol | Eye drops | Clorocil | Europe |

Sulphobutylether -cyclodextrin

| | | | |
|---|---|---|---|
| Voriconazole | Intravenous | Vfend | Europe, |
| | | | United |
| | | | States |
| Ziprasidone maleate | Intramuscular | Geodon, Zeldox | Europe, |
| | | | United |
| | | | States |

2-Hydroxypropyl--cyclodextrin

| | | | |
|---|---|---|---|
| Diclofenac sodium | Eye drops | Voltaren | Europe |

### SUMMARY OF THE INVENTION

The present invention describes curcumin cyclodextrin complexes that exhibit enhanced bioavailability of curcumin and can be used as therapeutic agents to treat, and possibly slow or prevent a number of diseases and conditions. The composition can be provided as a gel, capsule, tablet, powder, liquid, or other pharmaceutically acceptable form.

The invention also provides a method of use for treating, slowing, and/or preventing diseases. The diseases include neurological diseases such as Alzheimers, autoimmune or inflammatory or allergic diseases such as asthma and rheumatoid arthritis, oncologic diseases, respiratory system diseases such as chronic obstructive lung disease, dermatologic diseases, cardiovascular diseases such as hyperlipidemia and coronary artery disease, metabolic diseases such as diabetes, gastrointestinal, hepatic and pancreatic diseases such as inflammatory bowel disease, urologic, and infectious diseases and for wound healing.

### DETAILED DESCRIPTION OF THE INVENTION

Cyclodextrin may be purchased from a number of vendors including Sigma-Aldrich, Inc. (St. Louis, Mo., USA). In addition, other forms of amorphous cyclodextrin having different degrees of substitution or glucose residue number are available commercially. A method for the production of hydroxypropyl-p-cyclodextrin is disclosed in Pitha et al., U.S. Pat. No. 4,727,064 which is incorporated herein by reference.

To produce the formulations according to the invention, a pre-weighed amount of cyclodextrin, which is substantially pyrogen free is placed in a suitable depyrogenated sterile container. Methods for depyrogenation of containers and closure components are well known to those skilled in the art and are fully described in the United States Pharmacopeia 23 (United States Pharmacopeial Convention, Rockville, Md. USA). Generally, depyrogenation is accomplished by exposing the objects to be depyrogenated to temperatures above 400° C. for a period of time sufficient to fully incinerate any organic matter. As measured in U.S. P. Bacterial Endotoxin Units, the formulation will contain no more than 10 Bacterial Endotoxin Units per gram of amorphous cyclodextrin. By substantially pyrogen free is meant that the hydroxypropyl-p-cyclodextrin contains less than 10 U.S.P. bacterial endotoxin units per gram using the U.S.P. method. Preferably, the hydroxypropyl-(3-cyclodextrin will contain between 0.1 and 5 U.S.P. bacterial endotoxin units per mg, under conditions specified in the United States Pharmacopeia 23.

Make a 20% HPβ-cyclodextrin solution, pH 11-12: Place 2 g HPβ-cyclodextrin (Sigma/Aldrich #332593) per 8 ml water in a stoppered graduated cylinder with at least a 50% volume reserve; shake vigorously to dissolve, then place in an ultrasonic bath briefly to clarify; vortex to ensure homogeneous mixing. Add a stir bar and 10% NaOH dropwise with stirring until pH is between 11 & 12; qs to 2 g of cyclodextrin/10 ml water and re-mix by vortexing. Use at room temperature.

Make a 15 mg/ml curcumin stock solution, pH 11-12: Suspend 15 mg of curcumin (Sigma C7727) per 0.9 ml of an ethanol/water (10/90) mix in a beaker (with a stir bar) that is large enough for a pH monitoring electrode. Add 10% NaOH dropwise while stirring and using a pipette to wet any remaining powder with liquid until the drug is well suspended and suspension turns red/purple. Insert pH electrode and continue adding NaOH until pH is between 11 and 12 and suspended drug is dissolved (clear purple). Then qs to 15 mg/1.0 ml and stir for ˜5 more minutes to make sure drug is in solution. Then add to cyclodextrin solution immediately (see 0052 below) as alkaline curcumin is unstable.

Make neutral 3 mg/ml Cyclodextrin: Curcumin (CDC) dosing solution: Add 2 ml of alkaline curcumin stock per 8 ml of alkaline cyclodextrin in a beaker with stir bar; curcumin should stay dissolved. Then complex the curcumin with the cyclodextrin by slowly lowering the pH via dropwise addition of 0.8 M citric acid with stirring and pH monitoring until pH is between 6 & 7. As pH drops the color will change to a clear orange/yellow.

Store drug in dark or amber vials. Use immediately or store in the refrigerator; the drug is still clear after overnight refrigeration.

The drug can be dried in a speed vac and stored at room temperature. Reconstitution can be achieved by mixing with water to the original concentration.

Efficacy: The efficacy of CDC was compared to native curcumin in a mouse paw edema model.
Purpose: To compare the oral efficacy of an aqueous curcumin suspension (C) with that of a curcumin cyclodextrin complex (CDC) in the mouse carrageenan paw inflammation model
Materials: Curcumin (C) (Sigma C7727, lot #096K1092); HPβ-Cyclodextrin (CD) (Sigma/Aldrich 332593, lot #04806CJ); Indomethacin (Sigma 18280, lot #098K1500); λCarrageenan (Fluka 22049, lot #1408463); ketamine (Bioniche Pharma NDC 67457-001-10); xylazine (Lloyd Labs, lot #LA33806); calipers (Fisher 15-077-957); ethanol (E) (Decon Labs #2716, lot #A01190801 L).
Animals: 35 male CD-1 mice, 20-30 g (Harlan)
Experimental Design/Methods/Groups:
   1. Acclimatize the mice for 3+ days after receipt. Weigh mice within 24 hrs of dosing to calculate dosing volume. Remove food 2 hours before dosing; restore food ˜2 hours after dosing.
   2. Vehicle and Drugs:
      Vehicle: Make 20 ml of 20% cyclodextrin (CD) by dissolving 4 g CD/20 ml sterile water. Make 10 ml of Ethanol/water (25%/75%) by mixing 2.5 ml of Ethanol and 7.5 ml sterile water. Make 5 ml of Ethanol/water (5%/95%). Separate CD into two 10 ml aliquots; add 10% NaOH dropwise to one of them until the pH is 11-12; adjust the other to pH 6-8 if needed. For the vehicle group, make 5 ml of vehicle by mixing 1 ml of Ethanol/Water (25%/75%) with 4 ml of 20% CD, pH 6-8. Store in fridge for up to 3 days.
      Curcumin: Make 5 ml of 3 mg/ml curcumin dosing suspension by suspending 15 mg of drug in 5 ml of Ethanol/Water (5%/95%); vortex and sonicate to make a fine suspension. Check ability to push through an appropriately sized gavage needle; if clumpy, homogenize and re-check. Store in fridge overnight protected from light.
      CDC: Make 5 ml of 15 mg/ml curcumin stock by suspending 75 mg in 5 ml of Ethanol/Water (25%/75%). Then add 10% NaOH dropwise with stirring until pH reaches 11-12 and drug is dissolved; stir 10 mins (solution will be red/purple). Then make 10 ml of dosing solution by taking 2 ml of dissolved drug and adding slowly to 8 ml of pH 11-12 20% CD solution with constant stirring; mix for 10 mins. Next complex the curcumin by slow dropwise addition of 0.8 M citric acid until pH is 6-7 and solution is clear yellow. Store in fridge overnight protected from light.
      Indomethacin: Make 10 ml of dosing solution by suspending 5 mg in 10 ml of sterile water with stirring, then adding 0.1 N NaCO3 dropwise until solid disappears and pH is 7-8. Store in fridge for up to 1 week.
   3. Carrageenan: Make 2 ml of 1% carrageenan by dissolving 20 mg in 2 ml of saline at 4° C. and then vortexing, warming and sonicating. Test for injection resistance by 27G and 28 G needles. Store in refrigerator overnight.
   4. Groups: See Group Variables table below.

Group Variables

| | | | | Dose | Dose | Dose |
|---|---|---|---|---|---|---|
| Group | No. of | Test | Dose | Level | Volume | Conc. |
| No. | Animals | Article | Route | (mg/kg) | (mL/kg) | (mg/mL) |
| 1 | 8 | Vehicle | p.o. | N/A | 10 | N/A |
| 2 | 8 | Curcumin | p.o. | 30 | 10 | 3 |
| 3 | 8 | CDC | p.o. | 30 | 10 | 3 |
| 4 | 8 | Indometha | p.o. | 5 | 10 | 0.5 |

5. Procedure: Measure thickness of each paw of each mouse.

Dose mice p.o. 30 minutes before carrageenan injection. Anesthetize with ketamine/xylazine (100/12 mg/kg, i.p.) 15 minutes before carrageenan injection. At t=0, inject left footpad of each mouse with 25 μL of 1% carrageenan, using a 27 or 28 G needle. At 3 hours, measure thicknesses of each paw with calipers, holding mouse vertical and paw out with forceps. Calculate edema by subtracting right paw thickness from left paw thickness.

Results: The paw was measured at 3 hours after oral gavage of curcumin (30 mg/kg), Indomethacin (5 mg/kg), and CDC (30 mg/kg) to mice. After 3 hours, the swelling was 0.59 mm for CDC, 0.66 mm for curcumin, 0.73 mm for indomethacin, and 0.78 mm for vehicle control. The difference between CDC and curcumin/indomethacin was statistically significant, with p<0.05.

The invention has been described with reference to various embodiments and examples, but is not limited thereto. Variations may be made without departing from the invention's scope, which is limited only by the appended claims and equivalents thereof.

### REFERENCES

### Curcumin

Yang F et al. Curcumin inhibits formation of amyloid beta oligomers and fibrils, binds plaques, and reduces amyloid in vivo. J Biol Chem, Vol. 280, No. 7. (18 Feb. 2005), pp. 5892-5901.
Hatcher H, Planalpb R, Chob J, Tortia,d F M, and Tortic,d S V. Curcumin: From ancient medicine to current clinical trials. Cell. Mol. Life Sci. 65 (2008) 1631-1652.
Aggarwal B B, Kumar A, Bharti A C. Anticancer potential of curcumin: preclinical and clinical studies. Anticancer Res. 2003 January-February; 23(1A):363-98.
Cheng, A L, Hsu, C H, Lin, J K, Hsu, M M, Ho, Y F, Shen, T S, Ko, J Y, Lin, J T, Lin, B R, Ming-Shiang, W, Yu, H S, Jee, S H, Chen, G S, Chen, T M, Chen, C, Lai, M K, Pu, Y S, Pan, M H, Wang, Y J, Tsai, C C and, Hsieh, C Y. Phase I clinical trial of curcumin, a chemopreventive agent, in patients with high-risk or pre-malignant lesions. Anticancer Res 2001. 21, 2895-2900.
Hatcher H, Planalp R, Cho J, Torti F M and Torti S V. Curcumin: From ancient medicine to current clinical trials. Cell. Mol. Life Sci. 65 (2008) 1631-1652

Drug delivery and complexes (including cyclodextrins)
Davis M E and Brewster M E. Cyclodextrin-based pharmaceutics: past, present and future. Nature Reviews Drug Discovery 3, 1023-1035 (December 2004).
Muller B W, Waaler T, Helm H and Anderson A: Complex formation of beta and gamma cyclodextrins with digitoxin, Acta Pharm. Nord. 1992; 4: 313-317.
Albers E and Muller B W: Cyclodextrin derivatives in pharmaceutics, Crit Rev Ther Drug Carrier Syst. 1995; 12:311-37.
Uekama K, Fujinaga T, Hirayama F, Otagiri M, Yamasaki M, Seo H, Hashimoto T, Tsuruoka M: Improvement of the oral bioavailability of digitalis glycosides by cyclodextrin complexation, J Pharm Sci 1983; 72: 1338-41.
Okada Y, Koizumi K: Some properties and the inclusion behavior of three positional isomers of 6(1), 6n-di-O-alpha-D-glucosyl-cyclomaltoheptaoses (beta-cyclodextrins), Chem Pharm Bull (Tokyo) 1998; 46: 319-23.
Ueda H, Wakamiya A, Endo T, Nagase H, Tomono K, Nagai T: Interaction of cyclomaltononaose (delta-CD) with several drugs, Drug Dev Ind Pharm 1999; 25: 951-4.
Bekers et al.: “Stabilization of Mitomycins on Complexation With Cyclodextins in Aqueous Acidic Media”, Intl. J. of Pharmaceutics, 52, 239 (1989).
Allen T M, Hansen C, Martin F J, Redemann C and Yau-Young A: Liposome containing a synthetic lipid derivative of poly(ethylene glycol) show prolonged circulation half-lives in vivo. Biochim. Biophys. Acta, 1066, 29-36 (1991).
Chopra R N, Nayar S L and Chopra I C: Glossary of Indian Medicinal Plants, CSIR, New Delhi (1956), page 175.
Dey K L and Bahadur R: Indigenous Drugs of India, International Book Publishers, India (1984).
Drummond D C et al: Optimizing Liposomes for Delivery of Chemotherapeutic Agents to Solid Tumors. Pharmacological Reviews 51, 691-743, 1999 and the references cited therein.
Dymock W: Pharmacographia Indica, 2, 398, (1890).
Gabizon A. and Papahadjopoulos D: Liposome formulations with prolonged circulation time in blood and enhanced uptake by tumors. Proc. Natl. Acad. Sci. (Wash.) 85, 6949-53 (1988).
Lee et al: “Serum Albumin Beads: An Injectable, Biodegradable System for the Sustained Release of Drugs” Science, 213:233-235 (1981).
Leucuta et al: “Albumin microspheres as a drug delivery system for epirubicin: pharmaceutical, pharmacokinetic and biological aspects” International Journal of Pharmaceutics, 41:213-217 (1988).
Madden T D, Harrigan P R, Tai L C L, Bally M B, Mayer L D, Redelmeier T E, Loughrey H C, Tilcock C P S, Reinish L W and Cullis P R: The accumulation of drugs within large unilamellar vesicles exhibiting a proton gradient: a survey, Chem. Phys. Lipids, 53, 37-46 (1990).
William S S, Alosco T R, Mayhew E, Lasic D D, Martin F J and Bankert R B: Arrest of human lung tumor xenograft growth in severe combined immunodeficient mice using doxorubicin encapsulated in sterically stabilized liposomes. Cancer Res., 53, 3964-67 (1993).
Repke K R H, Schonfeld W, Weiland J, Megges R, Hache, A: In Design of Enzyme Inhibitors as Drugs; Sandler, M.; Smith, H. J., Eds; Oxford University Press: Oxford, 1989; pp 435-502.
Thomas R E, Gray P, Andrews J: In Advances in Drug Research; Testa, L. B., Ed.; Academic Press: London, 1990; Vol. 19, pp 313-575.
Thomas R E: In Molecular Structure and Biological Activity of Steroids; Bohl M, Daux W L, Eds.; CRC Press: Boca Raton, 1992; pp 399-464.
Cassady J M, Suffness M M: Terpenoid antitumor targets. In: Anti cancer agents based on natural product models (Edited by Cassady J M, Douros J D) New York, Academic Press 1980, 201-269.
Mitsuhashi H, Tanaka O, Nozoe S, Nagai M. (ed.): Chemistry of Organic Natural Products. 4th ed, Nankoudou Press, Tokyo, 1994, 168-174.

NFkB
Abraham E (2003) Nuclear factor-kB and its role in sepsis-associated organ failure. Journal of Infectious Diseases 187: S364-S369
Aradhya S and Nelson D L (2001) NF-kB signaling and human disease. Current Opinions in Genetics and Development 11: 300-306.
Arima N and Tei C (2001) HTLV-I Tax related dysfunction of cell cycle regulators and oncogenesis of adult T cell leukemia. Leukemia and Lymphoma 40: 267-278
Baghdiguian S, Martin M, Richard I, Pons F, Astier C, Bourg N, Hay R T, Chemaly R, Halaby G, Loiselet J, Anderson L V, Lopez de Munain A, Fardeau M, Mangeat P, Beckmann J S and Lefranc G (1999) Calpain 3 deficiency is associated with myonuclear apoptosis and profound perturbation of the IkB alpha/NF-kB pathway in limb-girdle muscular dystrophy type 2A [published erratum appears in Nat Med July 1999; 5(7):849]. Nature Medicine 5: 503-511
Baldwin A S Jr (2001) Series introduction: the transcription factor NF-kB and human disease. Journal of Clinical Investigation 107: 3-6
Bargou R C, Leng C, Krappmann D, Emmerich F, Mapara M Y, Bommert K, Royer H-D, Scheidereit C and Dorken (1996) High-level nuclear NF-kB and October-2 is a common feature of cultured Hodgkin/Reed-Sternberg cells. Blood 87: 4340-4347
Bargou R C, Emmerich F, Krappmann D, Bommert K, Mapara M Y, Arnold W, Royer H D, Grinstein E, Greiner A, Scheidereit C and Dorkin B (1997) Constitutive NF-kB-RelA activation is required for proliferation and survival of Hodgkin's disease tumor cells. Journal of Clinical Investigation 100: 2961-2969
Barnes P J (2002) New treatments for COPD. Nature Reviews Drug Discovery 1: 437-446
Baumann B, Weber C K, Troppmair J, Whiteside S, Israel A, Rapp U R and Wirth T (2000) Raf induces NF-kB by membrane shuttle kinase MEKK1, a signaling pathway critical for transformation. Proceedings of the National Academy of Sciences USA 97: 4615-4620
Bell S, Degitz K, Quirling M, Jilg N, Page S and Brand K (2003) Involvement of NF-kB signalling in skin physiology and disease. Cell Signal 15:1-7
Berenson J R, Ma H M and Vescio R (2001) The role of nuclear factor-kB in the biology and treatment of multiple myeloma. Seminars in Oncology 28: 626-633
Besancon F, Atfi A, Gespach C, Cayre Y E and Bourgeade M F (1998) Evidence for a role of NF-kB in the survival of hematopoietic cells mediated by interleukin-3 and the oncogenic TEL/platelet-derived growth factor beta fusion protein. Proceedings of the National Academy of Sciences USA 95: 8081-8086
Bian X, Opipari Jr A W, Ratanaproeksa A B, Boltano A E, Lucas P C and Castle V P (2002) Constitutively active NF-kB is required for the survival of S-type neuroblastoma. Journal of Biological Chemistry 277: 42144-42150
Bielory L, Kempuraj D and Theoharides T (2003) Topical immunopharmacology of ocular allergies. Current Opinion in Allergy and Clinical Immunology 2: 435-445
Brummelkamp T R, Nijman S M B, Cirac A M G and Bernards R (2003) Loss of the cylindromatosis tumour suppressor inhibits apoptosis by activating NF-kB. Nature 424 797-801
Cechetto D F (2001) Role of nuclear factor kappaB in neuropathological mechanisms. Progress in Brain Research 132: 391-404 [0295]
Chen F and Shi X (2002) NF-kB, a pivotal transcription factor in silica-induced diseases. Mol Cell Biochem 234-235: 169-176
Christman J W, Sadikot R T and Blackwell T S (2000) The role of nuclear factor-kB in pulmonary diseases. Chest 117 1482-1487
Chung H Y, Kim H J, Kim K W, Choi J S and Yu B P (2002) Molecular inflammation hypothesis of aging based on the anti-aging mechanism of calorie restriction. Microsc Res Tech 59: 264-272
Courtois G and Israel A (2000) NF-kB defects in humans: the NEMO/incontinentia pigmenti connection. Sci STKE November 14 (58): PE1
Davis R E, Brown K D, Siebenlist U and Staudt L M (2001) Constitutive nuclear factor kB activity is required for survival of activated B cell-like diffuse large B cell lymphoma cells. Journal of Experimental Medicine 194: 1861-1874
Dejardin E, Deregowski V, Chapelier M, Jacobs N, Gielen J, Merville M-P and Bours (1999) Regulation of NF-kB activity by IkB-related proteins in adenocarcinoma cells. Oncogene 18: 2567-2577
Dijkstra G, Moshage H and Jansen P L (2002) Blockade of NF-kB activation and donation of nitric oxide: new treatment options in inflammatory bowel disease? Scandinavian Journal of Gastroenterology Suppl 236: 37-41
Finco T S, Westwick J K, Norris J L, Beg A A, Der C J and Baldwin Jr A S (1997) Oncogenic Ha-Ras-induced signaling activates NF-kB transcriptional activity, which is required for cellular transformation. Journal of Biological Chemistry 272: 24113-24116
Furman R R, Asgary Z, Mascarenhas J O, Liou H C and Schattner E J (2000) Modulation of NF-kB activity and apoptosis in chronic lymphocytic leukemia B cells. Journal of Immunology 164: 2200
Gilmore T D, Gapuzan M-E, Kalaitzidis D and Starczynowski D (2002) Rel/NF-kB/IkB signal transduction in the generation and treatment of human cancer. Cancer Letters 181: 1-9
Guijarro C and Egido J (2001) Transcription factor-kappaB (NF-kB) and renal disease. Kidney International 59: 415-424
Guzman M L, Neering S J, Upchurch D, Grimes B, Howard D S, Rizzieri D A, Luger S M, Jordan C T (2001) Nuclear factor-kB is constitutively activated in primitive human acute myelogenous leukemia cells. Blood 98: 2301-2307
Hayashi T and Faustman D (2002) Defective function of the proteasome in autoimmunity: involvement of impaired NF-kB activation. Diabetes Technol Ther 2: 415-428
Hayashi S, Yamamoto M, Ueno Y, Ikeda K, Ohshima K, Soma G and Fukushima T (2001) Expression of nuclear factor-kB, tumor necrosis factor receptor type 1, and c-Myc in human astrocytomas. Neurologia Medico-Chirufica 41: 187-195
Hiscott J, Kwon H and Genin P (2001) Hostile takeovers: viral appropriation of the NF-kB pathway. Journal of Clinical Investigation 107: 143-151
Ho E and Bray T M (1999) Antioxidants, NFkB activation, and diabetogenesis. Proc Soc Exp Biol Med 222: 205-213
Holmes-McNary M (2002) Nuclear factor kappaB signaling in catabolic disorders. Curr Opin Clin Nutr Metab Care 5: 255-263
Huang S, Pettaway C A, Uehara H, Bucana C D and Fidler I J (2001) Blockade of NF-kB activity in human prostate cancer cells is associated with suppression of angiogenesis, invasion, and metastasis. Oncogene 20: 4188-4197
Huang S, Robinson J B, DeGuzman A, Bucanna C D and Fidler I J (2000) Blockade of nuclear factor-kB signaling inhibits angiogenesis and tumorigenicity of ovarian cancer cells by suppressing expression of vascular endothelial growth factor and Interleukin 8. Cancer Research 60: 5334-5339.
Lavie L (2003) Obstructive sleep apnoea syndrome—an oxidative stress disorder. Sleep Medicine Review 7: 35-51 [0315] 151. Karin M, Cao Y, Greten F R and Li Z W (2002) NF-kB in cancer: from innocent bystander to major culprit. Nature Reviews Cancer 2: 301-310
Kammer G M and Tsokos G C (2002) Abnormal T lymphocyte signal transduction in systemic lupus erythematosus. Current Directions in Autoimmunity 5: 131-150
Knecht H, Berger C, Rothenberger S, Odermatt B F and Brousset P (2001) The role of Epstein-Barr virus in neoplastic transformation. Oncology 60: 289-302
Kordes U, Krappmann D, Heissmeyer V, Ludwig W D and Scheidereit C (2000) Transcription factor NF-kB is constitutively active in acute lymphoblastic leukemia cells. Leukemia 14: 399-402
Kovalenko A, Chabie-Bessia C, Cantarella G, Israel A, Wallach D and Courtois G (2003) The tumour suppressor CYLD negatively regulates NF-kB signalling by deubiquitination. Nature 424: 801-805
Lind D S, Hochwald S N, Malaty J, Rekkas S, Hebig P, Mishra G, Moldawer L L, Copeland E M 3.sup.rd and Mackay S (2001) Nuclear factor-kB is upregulated in colorectal cancer. Surgery 130: 363-369
Madjdpour L, Kneller S, Booy C, Pasch T, Schimmer R C and Beck-Schimmer B (2003) Acid-induced lung injury: role of Nuclear Factor-kB. Anesthesiology 99: 1323-1332
Martinez N, Camacho F I, Algara P, Rodriguez A, Dopazo A, Ruiz-Ballesteros E, Martin P, Martinez-Climent J A, Garcia-Conde J, Menarguez J, Solano F, Mollejo M and Piris M A (2003) The molecular signature of mantle cell lymphoma reveals multiple signals favoring cell survival. Cancer Research 63: 8226-8232
Mattson M P and Camandola S (2001) NF-kB in neuronal plasticity and neurodegenerative disorders. Journal of Clinical Investigation 107: 247-254
Nair A, Venkatraman M, Maliekal T T, Nair B, Karunagaran D (2003) NF-kB is constitutively activated in high-grade squamous intraepithelial lesions and squamous cell carcinomas of the human uterine cervix. Oncogene 22: 50-58
Nakshatri H, Bhat-Nakshatri P, Martin D A, Goulet Jr R J and Sledge Jr G W (1997) Constitutive activation of NF-kB during progression of breast cancer to hormone-independent growth. Molecular and Cellular Biology 17: 3629-3639
Nakayama H, Ikebe T, Beppu M and Shirasuma K (2001) High expression levels of nuclear factor kB, IkB kinase b and Akt kinase in squamous cell carcinoma of the oral cavity. Cancer 92: 3037-3044
Neurath M F, Becker C and Barbulescu K (1998) Role of NF-kB in immune and inflammatory responses in the gut. Out 43: 856-860
Nichols T C, Fischer T H, Deliargyris E N and Baldwin Jr A S (2001) Role of nuclear factor-kappaB (NF-kB) in inflammation, periodontitis, and atherogenesis. Annals of Periodontology 6: 20-29
Ondrey F G, Dong G, Sunwoo J, Chen Z, Wolf J S, Crowl-Bancroft C V, Mukaida N and Van Waes C (1999) Constitutive activation of transcription factors NF-kB, AP-1, and NF-IL6 in human head and neck squamous cell carcinoma cell lines that express pro-inflammatory and pro-angiogenic cytokines. Molecular Carcinogenesis 26: 119-129
Oya M, Ohtsubo M, Takayanagi A, Tachibana M, Shimizu N and Murai, M (2001) Constitutive activation of NF-kB prevents TRAIL-induced apoptosis in renal cancer cells. Oncogene 20: 3888-3896
Pahl A and Szelenyi I (2002) Asthma therapy in the new millennium. Inflammation Research 51: 273-282 [0332] 168. Palayoor S T, Yourmell M Y, Calderwood S K, Coleman C N and Price B D (1999) Constitutive activation of IkB kinase b and NF-kB in prostate cancer cells is inhibited by ibuprofen. Oncogene 18: 7389-7394
Pena A S and Penate M (2002) Genetic susceptibility and regulation of inflammation in Crohn's disease. Relationship with the innate immune system. [Article in English, Spanish] Rev Esp Enferm Dig 94: 351-360
Pennington C, Dunn J, Li C, Ha T and Browder W (2000) Nuclear factor kB activation in acute appendicitis: a molecular marker for extent of disease? American Surgery 66: 914-918
Purcell N H and Molkentin J D (2003) Is nuclear factor kappaB an attractive therapeutic target for treating cardiac hypertrophy? Circulation 108 638-640
Reuter U, Chiarugi A, Bolay H and Moskowitz M A (2003) Nuclear factor-kB as a molecular target for migraine therapy. Headache 43: 426-427
Reuther J Y, Reuther G W, Cortez D, Pendergast A M and Baldwin Jr A S (1998) A requirement for NF-kB activation in Bcr-Abl mediated transformation. Genes & Development 12: 968-981
Ritchie M E (1998) Nuclear factor kappaB is markedly and selectively activated in humans with unstable angina pectoris. Circulation 98 1707-1713
Roshak A K, Callahan J F and Blake S M (2002) Small-molecule inhibitors of NF-kB for the treatment of inflammatory joint disease. Current Opinion in Pharmacology 2: 316-321
Roshak A K, Callahan J F, Blake S M, Ross J S, Stagliano N E, Donovan M J, Breitbart R E and Ginsburg G S (2001) Atherosclerosis: a cancer of the blood vessels? American Journal of Clinical Pathology 116: S97-S107
Russell J P, Shinohara S, Melillo R M, Castellone M D, Santoro M and Rothstein J L (2003) Tyrosine kinase oncoprotein, RET/PTC3, induces the secretion of myeloid growth and chemotactic factors. Oncogene 22: 4569-4577
Santos S C, Monni R, Bouchaert I, Bernard O, Gisselbrecht S, Gouilleux F, Penard-Lacronique V (2001) Involvement of the NF-kB pathway in the transforming properties of the TEL-Jak2 leukemogenic fusion protein. FEBS Letters 497: 148-152
Sasaki N, Morisaki T, Hashizume K, Yao T, Tsuneyoshi M, Noshiro H, Nakamura K, Yamanaka T, Uchiyama A, Tanaka M and Katano M (2001) Nuclear factor-kB p65 (ReIA) transcription factor is constitutively activated in human gastric carcimoma tissue. Clinical Cancer Research 7: 4136-4142
Sclabas G M, Fujioka S, Schmidt C, Evans D B and Chiao P J (2003) NF-kB in pancreatic cancer. International Journal of Gastrointestinal Cancer 33 15-26
Seppanen M and Vihko K K (2000) Activation of transcription factor NF-kB by growth inhibitory cytokines in vulvar carcinoma cells. Immunology Letters 74: 103-109
Shaffer A L, Rosenwald A and Staudt L M (2002) Lymphoid malignancies: the dark side of B-cell differentiation. Nature Review Immunology 2: 920-933
Sovak M A, Bellas R E, Kim D W, Zanieski G J, Rogers A E, Traish A M and Sonenshein G E (1997) Aberrant nuclear factor-kB/Rel expression and the pathogenesis of breast cancer. Journal of Clinical Investigation 100: 2952-2960
Staudt L M (2000) The molecular and cellular origins of Hodgkin's disease. Journal of Experimental Medicine 191: 207-212
Sutter A P, Zeitz M and Scherubl H (2004) Recent results in understanding molecular pathways in the medical treatment of esophageal and gastric cancer. Onkologie 27 17-21
Tai D I, Tsai S L, Chang Y H, Huang S N, Chen T C, Chang K S and Liaw Y F (2000) Constitutive activation of nuclear factor kB in hepatocellular carcinoma. Cancer 89: 2274-2281
Toledo-Pereyra L and Lopez-Neblina F (2002) New advances in the molecular biology of ischemia/reperfusion: MAPK pathways. Tissue Antigens 60: 552
Trompouki E, Hatzivassillou E, Tsichritzis T, Farmer H, Ashworth A and Mosialos G (2003) CYLD is a deubiquitinating enzyme that negatively regulates NF-kB activation by TNFR family members. Nature 424 793-796
Valen G, Yan Z Q and Hansson G K (2001) Nuclear factor kB and the heart. Journal of the American College of Cardiology 38: 307-314
Visconti R, Cerutti J, Battista S, Fedele M, Trapasso F, Zeki K, Miano M P, de Nigris F, Casalino L, Curcio F, Santoro M and Fusco A (1997) Expression of the neoplastic phenotype by human thyroid carcinoma cell lines requires NFkB p65 protein expression. Oncogene 15: 987-1994
Wang W, Abbruzzese J L, Evans D B, Larry L, Cleary K and Chiao P J (1999) The NF-kB ReIA transcription factor is constiutively activated in human pancreatic adenocarcinoma cells. Clinical Cancer Research 5: 119-127
Weber C K and Adler G (2001) From acinar cell damage to systemic inflammatory response: current concepts in pancreatitis. Pancreatology 1: 356-362
Whitehead I P, Lambert Q T, Glaven J A, Abe K, Rossman K L, Mahon G M, Trzaskos J M, Kay R, Campbell S L and Der C J (1999) Dependence of Dbl and Dbs transformation on MEK and NF-kB activation. Molecular and Cellular Biology 19: 7759-7770
Wilson S H, Caplice N M, Simari R D, Holmes D R, Jr., Carlson P J and Lerman A (2000) Activated nuclear factor-kB is present in the coronary vasculature in experimental hypercholesterolemia. Atherosclerosis 148: 23-30
Wratten M L, Brendolan A, Ronco C, La Greca G and Tetta C (2001) Should we target signal pathways instead of single mediators in the treatment of sepsis? Contrib Nephrol 132: 400-414
Yamamoto Y and Gaynor R B (2001) Role of the NF-kB pathway in the pathogenesis of human disease states. Curr Mol Med 1: 287-296 [0361] 197. Yang C W, Wu M S and Pan M J (2001) Leptospirosis renal disease. Nephrol Dial Transplant 16 Suppl 5: 73-77
Yang J and Richmond A (2001) Constitutive IkB kinase activity correlates with nuclear factor-kB activation in human melanoma cells. Cancer Research 61: 4901-4909
Yuan M, Korzstantopoulos N, Lee J, Hansen L, Li Z W, Karin M and Shoelson S E (2001) Reversal of obesity- and diet-induced insulin resistance with salicylates or targeted disruption of IKKb. Science 293: 1673-1677.

What is claimed:

1. A curcumin cyclodextrin complex having enhanced bioavailability and/or efficacy, irrespective of the ratio of curcumin and cyclodextrin

2. A curcumin composition as recited in claim 1, wherein the curcumin is selected from the group consisting of curcumin, tetrahydrocurcumin, demethoxycurcumin, bisdemethoxycurcumin, curcumin esters, and mixtures thereof.

3. A curcumin composition as recited in claim 1, wherein the curcuminoid comprises a curcumin pro-drug.

4. A cyclodextrin as recited in claim 1, wherein the cyclodextrin is alpha, beta, or gamma cyclodextrin.

5. A cyclodextrin as recited in claim 1, wherein the cyclodextrin may be amorphous or crystalline.

6. A curcumin cyclodextrin complex as recited in claim 1, wherein the complex has the form of a solid powder, liquid, microemulsion, solid lipid nanoparticles, microencapsulated oil, or gel, and is suitable for parenteral, topical or enteral administration.

7. A method of use of the complex as recited in claim 1 to treat diseases including but not limited to neurological diseases such as Alzheimers, autoimmune or inflammatory or allergic diseases such as asthma and rheumatoid arthritis, oncologic diseases, respiratory system diseases such as chronic obstructive lung disease, dermatologic diseases, cardiovascular diseases such as hyperlipidemia and coronary artery disease, gastrointestinal hepatic and pancreatic diseases such as inflammatory bowel disease, metabolic diseases such as diabetes, urologic, infectious diseases and for wound healing.

8. A method of use as recited in claim 1 wherein the administration can be oral, topical, parenteral, nasal, inhalational, or suppository.

9. A method of use as recited in claim 1 where the complex is administered concurrently with any therapy for the treatment or prevention of diseases as mentioned in 7.

10. A method of use as recited in claim 1 where the complex is used with a device such as coating a coronary artery stent.
